(19) United States
(12) Patent Application Publication
(10) Pub. No.: US 2010/0270537 A1
(43) Pub. Date: Oct. 28, 2010

(54) OPTOELECTRONIC DEVICES AND ORGANIC COMPOUNDS USED THEREIN

(75) Inventors: Qing Ye, Los Gatos, CA (US); James Anthony Cella, Clifton Park, NY (US); Jie Liu, Niskayuna, NY (US); Eisaku Katoh, Tokyo (JP)

Correspondence Address: GENERAL ELECTRIC COMPANY;GLOBAL RESEARCH, ONE RESEARCH CIRCLE, BLDG. K1-3A59, NISKAYUNA, NY, 12309, US

(73) Assignee: GENERAL ELECTRIC COMPANY, Schenectady, NY (US)

(21) Appl. No.: 12/428,055

(22) Filed: Apr. 22, 2009

(51) Int. Cl. H01L 51/54; C07D 471/14

(52) U.S. Cl. 257/40; 546/82; 257/E51.026

(57) ABSTRACT

An optoelectronic device comprises a cathode; an electron-transporting layer comprising a compound of formula I;

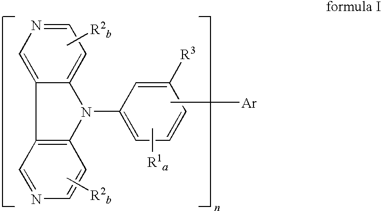

a light emitting-layer; and an anode; wherein R¹ and R² are independently at each occurrence, hydrogen, a C₁-C₂₀ aliphatic radical, a C₃-C₂₀ aromatic radical, or a C₃-C₂₀ cycloaliphatic radical; R³ is H or alkyl; a and b are, independently at each occurrence 0, or an integer ranging from 1 to 3; n is 2 or 3; and Ar is an aryl.

### BACKGROUND

The invention relates generally to optoelectronic devices and compounds used therein as, e.g., electron-transporting materials.

Optoelectronic devices, e.g. Organic Light Emitting Devices (OLEDs), which make use of thin film materials that emit light when subjected to a voltage bias, are expected to become an increasingly popular form of flat panel display technology. This is because OLEDs have a wide variety of potential applications, including cell phones, personal digital assistants (PDAs), computer displays, informational displays in vehicles, television monitors, as well as light sources for general illumination. Due to their bright colors, wide viewing angle, compatibility with full motion video, broad temperature ranges, thin and conformable form factor, low power requirements and the potential for low cost manufacturing processes, OLEDs are seen as a future replacement technology for cathode ray tubes (CRTs) and liquid crystal displays (LCDs). Due to their high luminous efficiencies, OLEDs are seen as having the potential to replace incandescent, and perhaps even fluorescent, lamps for certain types of applications.

OLEDs possess a sandwiched structure, which consists of one or more organic layers between two opposite electrodes. For instance, multi-layered devices usually comprise at least three layers: a hole injection/transport layer, an emissive layer and an electron transport layer (ETL). Furthermore, it is also preferred that the hole injection/transport layer serves as an electron blocking layer and the ETL as a hole blocking layer. Single-layered OLEDs comprise only one layer of materials between two opposite electrodes.

### BRIEF DESCRIPTION

In one aspect, the invention relates to an optoelectronic device comprising:
a cathode;
an electron-transporting layer comprising a compound of formula I;

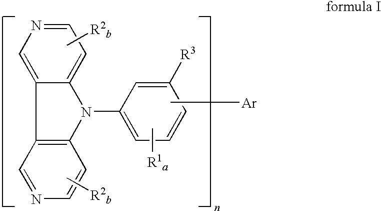

a light emitting-layer; and
an anode;
   wherein
R¹ and R² are independently at each occurrence, hydrogen, a C₁-C₂₀ aliphatic radical, a C₃-C₂₀ aromatic radical, or a C₃-C₂₀ cycloaliphatic radical;
R³ is H or alkyl;
a and b are, independently at each occurrence 0, or an integer ranging from 1 to 3;
n is 2 or 3; and
Ar is an aryl.

In another aspect, the invention relates to an organic compound of formula I.

### DETAILED DESCRIPTION

Organic compounds of formula I have properties suitable for use in electron-transporting layers of optoelectronic devices, e.g., organic light emitting devices (OLEDs) or photovoltaic devices.

In one aspect, the present invention relates to compounds of formula II, and optoelectronic devices having an electron-transporting layer containing these compounds

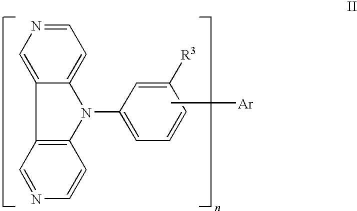

In another aspect, the present invention relates to compounds of formula III, and optoelectronic devices having an electron-transporting layer containing these compounds

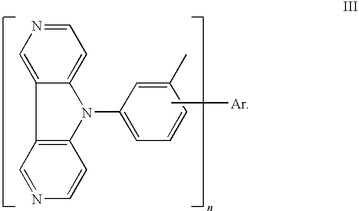

In yet another aspect, the present invention relates to compounds of formula IV, and optoelectronic devices having an electron-transporting layer containing these compounds.

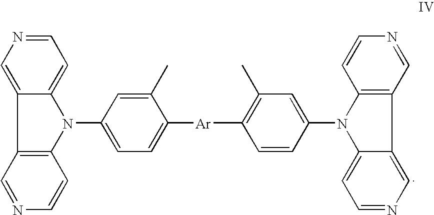

In yet another aspect, the present invention relates to compounds of formula V, and optoelectronic devices having an electron-transporting layer containing these compounds.

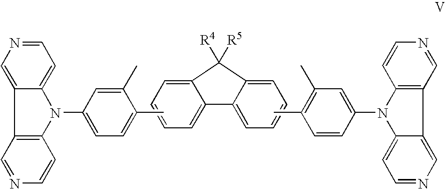

wherein R⁴ and R⁵ are independently a C₁-C₂₀ aliphatic radical, a C₃-C₂₀ aromatic radical, a C₃-C₂₀ cycloaliphatic radical, or R⁴ and R⁵, taken together, form a 10- to 13-membered bicyclic or tricyclic aromatic or heteroaromatic ring system.

According to some aspects of the invention, R⁴ and R⁵ may independently be a C₆-C₂₀ aliphatic radical, a C₆-C₂₀ aromatic radical, or a C₆-C₂₀ cycloaliphatic radical, or R⁴ and R⁵, taken together, form a 10- to 13-membered bicyclic or tricyclic aromatic or heteroaromatic ring system.

In yet another aspect, the present invention relates to compounds of formula VI, and optoelectronic devices having an electron-transporting layer containing these compounds.

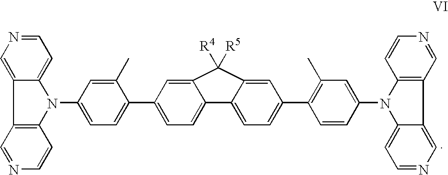

For the compounds of formula I to IV, Ar may be

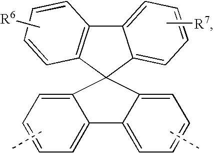

wherein R⁶ and R⁷ are independently a C₁-C₂₀ aliphatic radical, a C₃-C₂₀ aromatic radical, or a C₃-C₂₀ cycloaliphatic radical.

In some embodiments, Ar may be

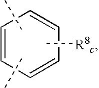

wherein R⁸ is independently at each occurrence a direct bond or an alkyl and c is an integer ranging from 2 to 6.

In some embodiments, Ar is independently chosen from

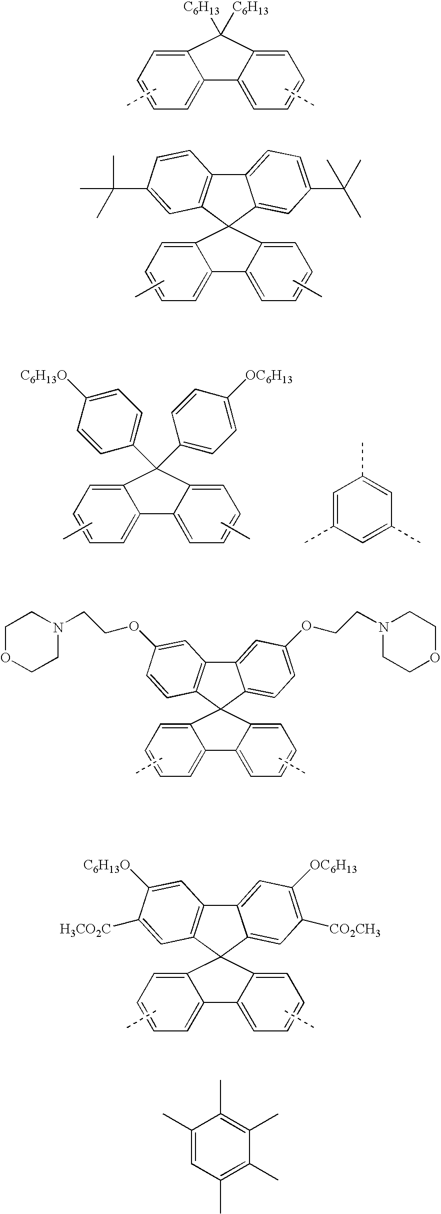

Examples of groups that may be used as Ar include

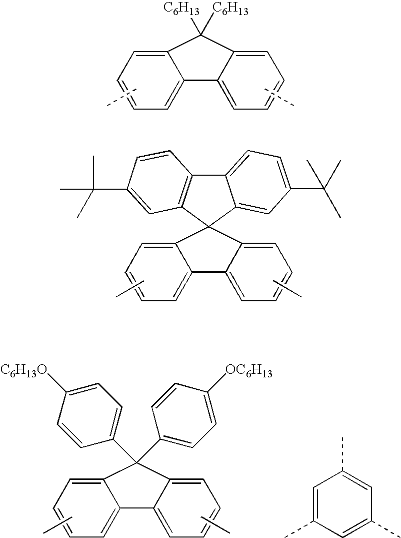

In some embodiments, R³ may be methyl. Examples of the organic compounds of formula I to VI include:

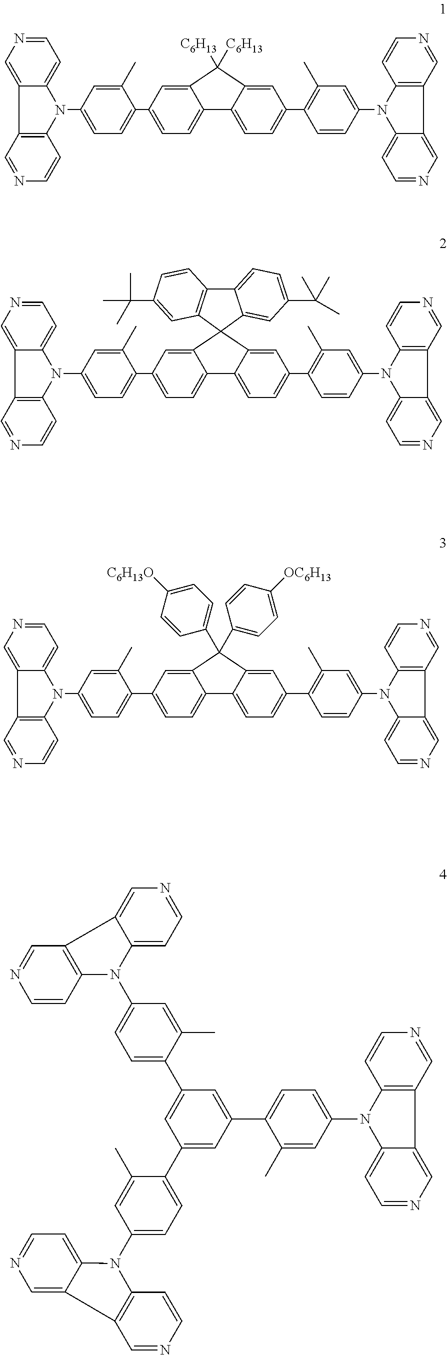

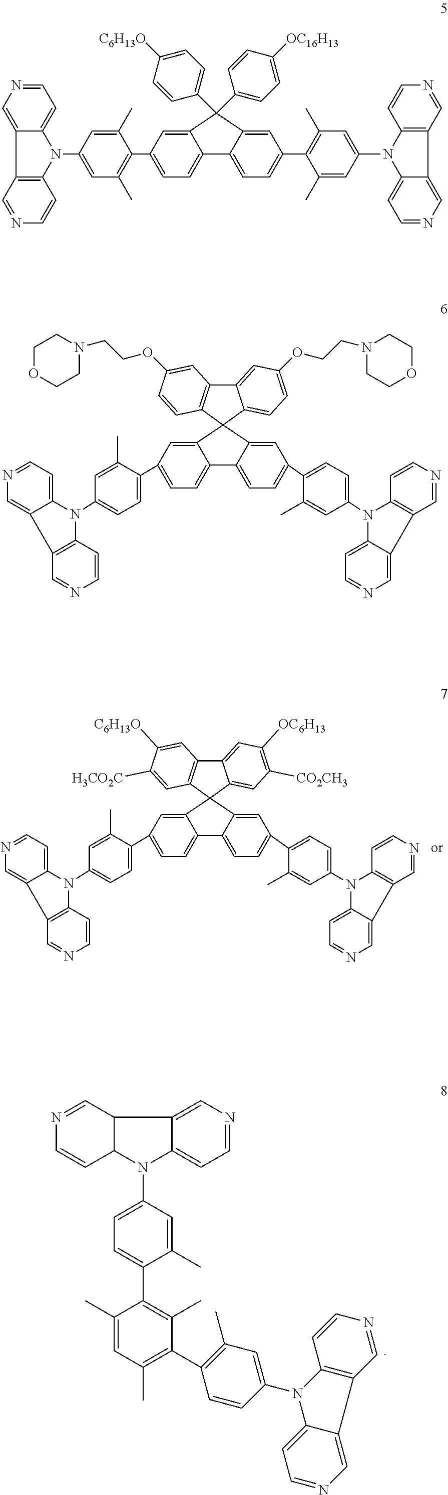

In particular embodiments, the organic compounds of formulas I to VI are chosen from

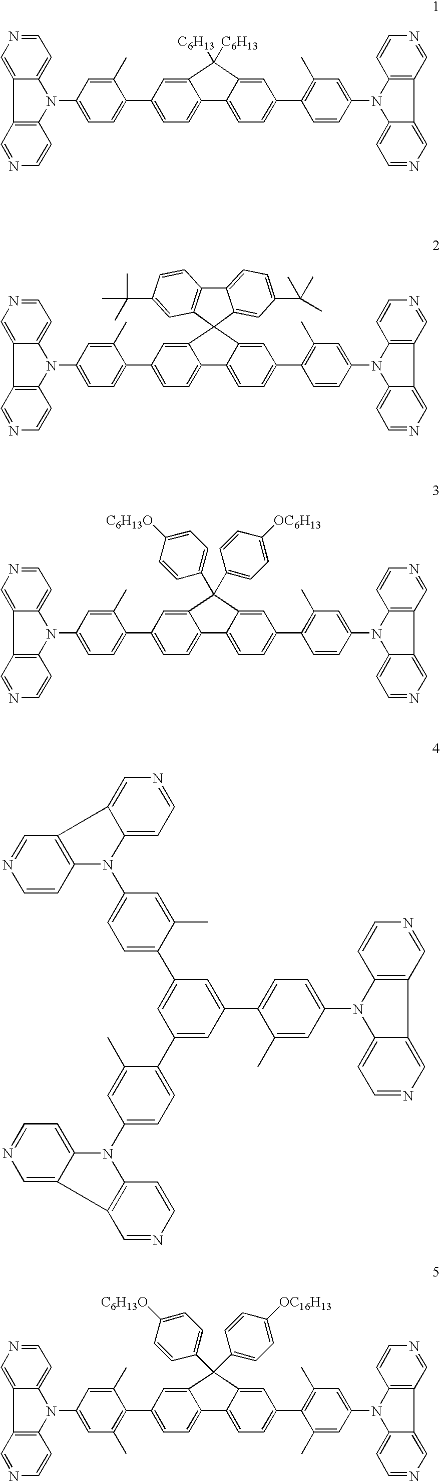

The compounds of formula I to VI may be prepared by employing Suzuki cross-coupling reactions. General procedure for Suzuki cross-coupling reactions includes mixing an aryl halide and aryl borate (or boronic acid) in a suitable solvent, in the presence of a base and Pd catalyst. The reaction mixture is heated under an inert atmosphere for a period of time. Suitable solvents include but are not limited to Dioxane, THF, EtOH, toluene and mixtures thereof. Exemplary bases include Na₂CO₃, K₂CO₃, Cs₂CO₃, Potassium phosphate and hydrates thereof. The bases can be added to the reaction as a solid powder or as an aqueous solution. The most commonly used catalysts include Pd(PPh₃)₄, or Pd(OAc)₂, Pd(dba)₂ with the addition of a secondary ligand. Exemplary ligands include dialkylphosphinobiphenyl ligands, such as structures VII-XI shown below, in which Cy is cyclohexyl.

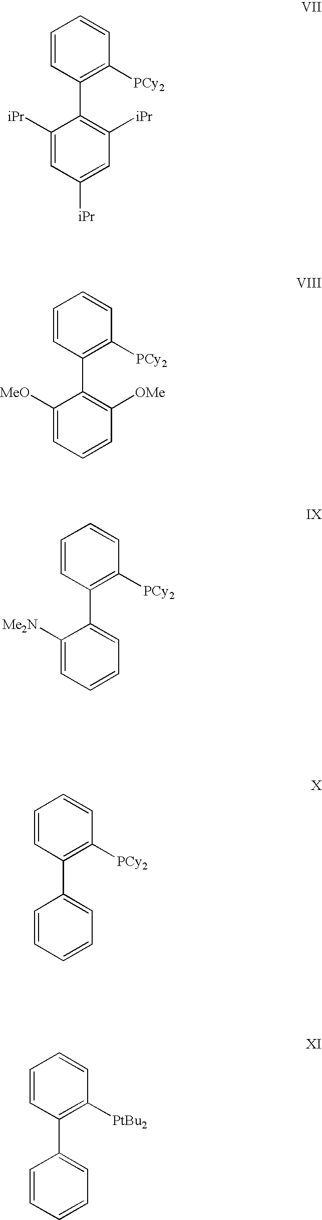

In general there are at least two methods that may be used to convert an aryl halide into its corresponding aryl borate. One method involves the generation of a carbanion using either BuLi to effect a lithio-halogen exchange or by using Mg to generate a Grignard reagent, followed by quenching of the carbanion with a borate such as trimethylborate, triethylborate or tri(isopropyl)borate and the like. A typical procedure involves combining the starting materials in dry solvents such as THF or diethylether under anhydrous and inert conditions. The reaction is cooled to −100° C. or −80° C. and BuLi is added dropwise and stirred at this temperature for a certain period of time (1-5 hours). After which time the intermediate carbanion is quenched with a suitable borate ester. The reaction mixture is allowed to warm to room temperature (RT), and, after stirring for 30 minutes at RT, the mixture is treated with a solution of saturated NH₄Cl (0.5 mL) and concentrated to dryness to afford a crude product.

The second method employs Pd-catalyzed borylation. A typical procedure includes combining an aryl halide and pinacolate diborane, anhydrous base and a dialkylphosphinobiphenyl ligand under dry and inert atmospheric conditions. The flask is protected from the atmosphere and charged with anhydrous solvent. After the solution is degassed for 15-30 minutes, the reaction mixture is charged with Pd catalyst and heated at reflux for an extended period by monitoring the disappearance of the starting aryl halide. Afterwards, the reaction mixture is cooled to RT and filtered. Upon concentration, the crude reaction product is afforded. Exemplary anhydrous bases include NaHCO₃, KHCO₃, and KOAc. Exemplary ligands include dialkylphosphinobiphenyl ligands, such as structures VII-XI and trans-dichlorobis(triphenylpho sphine)palladium(II).

An optoelectronic device, e.g., an OLED or a photovoltaic device, typically includes in the simplest case, an anode layer and a corresponding cathode layer with an organic electroluminescent layer disposed between said anode and said cathode. When a voltage bias is applied across the electrodes, electrons are injected by the cathode into the electroluminescent layer while electrons are removed from (or “holes” are “injected” into) the electroluminescent layer from the anode. Light emission occurs as holes combine with electrons within the electroluminescent layer to form singlet or triplet excitons, light emission occurring as singlet and/or triplet excitons decay to their ground states via radiative decay.

Other components which may be present in an OLED in addition to the anode, cathode and light emitting material include a hole injection layer, an electron injection layer, and an electron transport layer. The electron transport layer need not be in direct contact with the cathode, and frequently the electron transport layer also serves as a hole locking layer to prevent holes migrating toward the cathode. Additional components which may be present in an organic light-emitting device include hole transporting layers, hole transporting emission (emitting) layers and electron transporting emission (emitting) layers.

In one embodiment, an optoelectronic device of the invention may be a fluorescent OLED comprising a singlet emitter. In another embodiment, the OLEDs may be a phosphorescent OLED comprising at least one triplet emitter. In another embodiment, the OLEDs comprise at least one singlet emitter and at least one triplet emitter. The OLEDs may contain one or more, any or a combination of blue, yellow, orange, red phosphorescent dyes, including complexes of transition metals such as Ir, Os and Pt. In particular, electrophosphorescent and electrofluorescent metal complexes, such as those supplied by American Dye Source, Inc., Quebec, Canada may be used. Organic compounds of the formula I to VI may be part of electron transporting layer, or electron injection layer of an OLED.

The organic electroluminescent layer, i.e., the emissive layer, is a layer within an organic light emitting device which when in operation contains a significant concentration of both electrons and holes and provides sites for exciton formation and light emission. A hole injection layer is a layer in contact with the anode which promotes the injection of holes from the anode into the interior layers of the OLED; and an electron injection layer is a layer in contact with the cathode that promotes the injection of electrons from the cathode into the OLED; an electron transport layer is a layer which facilitates conduction of electrons from the cathode and/or the electron injection layer to a charge recombination site. During operation of an organic light emitting device comprising an electron transport layer, the majority of charge carriers (i.e. holes and electrons) present in the electron transport layer are electrons and light emission can occur through recombination of holes and electrons present in the emissive layer. A hole transporting layer is a layer which when the OLED is in operation facilitates conduction of holes from the anode and/or the hole injection layer to charge recombination sites and which need not be in direct contact with the anode. A hole transporting emission layer is a layer in which when the OLED is in operation facilitates the conduction of holes to charge recombination sites, and in which the majority of charge carriers are holes, and in which emission occurs not only through recombination with residual electrons, but also through the transfer of energy from a charge recombination zone elsewhere in the device. An electron transporting emission layer is a layer in which when the OLED is in operation facilitates the conduction of electrons to charge recombination sites, and in which the majority of charge carriers are electrons, and in which emission occurs not only through recombination with residual holes, but also through the transfer of energy from a charge recombination zone elsewhere in the device.

Materials suitable for use as the anode includes materials having a bulk resistivity of preferred about 1000 ohms per square, as measured by a four-point probe technique. Indium tin oxide (ITO) is frequently used as the anode because it is substantially transparent to light transmission and thus facilitates the escape of light emitted from electro-active organic layer. Other materials, which may be utilized as the anode layer, include tin oxide, indium oxide, zinc oxide, indium zinc oxide, zinc indium tin oxide, antimony oxide, and mixtures thereof.

Materials suitable for use as the cathode include general electrical conductors including, but not limited to metals and metal oxides such as ITO etc which can inject negative charge carriers (electrons) into the inner layer(s) of the OLED. Various metals suitable for use as the cathode include K, Li, Na, Cs, Mg, Ca, Sr, Ba, Al, Ag, Au, In, Sn, Zn, Zr, Sc, Y, elements of the lanthanide series, alloys thereof, and mixtures thereof. Suitable alloy materials for use as the cathode layer include Ag—Mg, Al—Li, In—Mg, Al—Ca, and Al—Au alloys. Layered non-alloy structures may also be employed in the cathode, such as a thin layer of a metal such as calcium, or a metal fluoride, such as LiF, covered by a thicker layer of a metal, such as aluminum or silver. In particular, the cathode may be composed of a single metal, and especially of aluminum metal.

Organic compounds of formula I to VI may be used in electron transport layers in place of, or in addition to traditional materials such as poly(9,9-dioctyl fluorene), tris(8-hydroxyquinolato)aluminum (Alq₃), 2,9-dimethyl-4,7-diphenyl-1,1-phenanthroline, 4,7-diphenyl-1,10-phenanthroline, 2-(4-biphenylyl)-5-(4-t-butylphenyl)-1,3,4-oxadiazole, 3-(4-biphenylyl)-4-phenyl-5-(4-t-butylphenyl)-1,2,4-triazole, 1,3,4-oxadiazole-containing polymers, 1,3,4-triazole-containing polymers, quinoxaline-containing polymers, and cyano-PPV.

Materials suitable for use in hole transporting layers include 1,1-bis((di-4-tolylamino)phenyl)cyclohexane, N,N′-bis(4-methylphenyl)-N,N′-bis(4-ethylphenyl)-(1,1′-(3,3′-dimethyl)biphenyl)-4,4′-diamine, tetrakis-(3-methylphenyl)-N,N,N′,N′-2,5-phenylenediamine, phenyl-4-N,N-diphenylaminostyrene, p-(diethylamino) benzaldehyde diphenylhydrazone, triphenylamine, 1-phenyl-3-(p-(diethylamino)styryl)-5-(p-(diethylamino)phenyl)pyrazoline, 1,2-trans-bis(9H-carbazol-9-yl)cyclobutane, N,N,N′,N′-tetrakis(4-methylphenyl)-(1,1′-biphenyl)-4,4′-diamine, copper phthalocyanine, polyvinylcarbazole, (phenylmethyl)polysilane; poly(3,4-ethylendioxythiophene) (PEDOT), polyaniline, polyvinylcarbazole, triaryldiamine, tetraphenyldiamine, aromatic tertiary amines, hydrazone derivatives, carbazole derivatives, triazole derivatives, imidazole derivatives, oxadiazole derivatives having an amino group, and polythiophenes as disclosed in U.S. Pat. No. 6,023,371.

Materials suitable for use in the light emitting layer include electroluminescent polymers such as polyfluorenes, preferably poly(9,9-dioctyl fluorene) and copolymers thereof, such as poly(9,9′-dioctylfluorene-co-bis-N,N′-(4-butylphenyl)diphenylamine) (F8-TFB); poly(vinylcarbazole) and polyphenylenevinylene and their derivatives. In addition, the light emitting layer may include a blue, yellow, orange, green or red phosphorescent dye or metal complex, or a combination thereof. Materials suitable for use as the phosphorescent dye include, but are not limited to, tris(1-phenylisoquinoline)iridium (III) (red dye), tris(2-phenylpyridine) iridium (green dye) and Iridium (III) bis(2-(4,6-difluorephenyl)pyridinato-N,C2) (blue dye). Commercially available electrofluorescent and electrophosphorescent metal complexes from ADS (American Dyes Source, Inc.) may also be used. ADS green dyes include ADS060GE, ADS061GE, ADS063GE, and ADS066GE, ADS078GE, and ADS090GE. ADS blue dyes include ADS064BE, ADS065BE, and ADS070BE. ADS red dyes include ADS067RE, ADS068RE, ADS069RE, ADS075RE, ADS076RE, ADS067RE, and ADS077RE.

Organic compounds of formula I to VI may form part of the electron transport layer. Thus, in one aspect, the present invention relates to more efficient optoelectronic devices, e.g., OLEDs comprising organic compounds of formula I to VI. The OLEDs may be phosphorescent containing one or more, any or a combination of, blue, yellow, orange, red phosphorescent dyes.

### DEFINITIONS

As used herein, the term “aromatic radical” refers to an array of atoms having a valence of at least one comprising at least one aromatic group. The array of atoms having a valence of at least one comprising at least one aromatic group may include heteroatoms such as nitrogen, sulfur, selenium, silicon and oxygen, or may be composed exclusively of carbon and hydrogen. As used herein, the term “aromatic radical” includes but is not limited to phenyl, pyridyl, furanyl, thienyl, naphthyl, phenylene, and biphenyl radicals. As noted, the aromatic radical contains at least one aromatic group. The aromatic group is invariably a cyclic structure having 4n+2 “delocalized” electrons where “n” is an integer equal to 1 or greater, as illustrated by phenyl groups (n=1), thienyl groups (n=1), furanyl groups (n =1), naphthyl groups (n=2), azulenyl groups (n=2), and anthraceneyl groups (n=3). The aromatic radical may also include nonaromatic components. For example, a benzyl group is an aromatic radical which comprises a phenyl ring (the aromatic group) and a methylene group (the nonaromatic component). Similarly a tetrahydronaphthyl radical is an aromatic radical comprising an aromatic group (C₆H₃) fused to a nonaromatic component —(CH₂)₄—. For convenience, the term “aromatic radical” is defined herein to encompass a wide range of functional groups such as alkyl groups, alkenyl groups, alkynyl groups, haloalkyl groups, haloaromatic groups, conjugated dienyl groups, alcohol groups, ether groups, aldehydes groups, ketone groups, carboxylic acid groups, acyl groups (for example carboxylic acid derivatives such as esters and amides), amine groups, nitro groups, and the like. For example, the 4-methylphenyl radical is a C₇ aromatic radical comprising a methyl group, the methyl group being a functional group which is an alkyl group. Similarly, the 2-nitrophenyl group is a C₆ aromatic radical comprising a nitro group, the nitro group being a functional group. Aromatic radicals include halogenated aromatic radicals such as 4-trifluoromethylphenyl, hexafluoroisopropylidenebis(4-phen-1-yloxy) (i.e., —OPhC(CF₃)₂PhO—), 4-chloromethylphen-1-yl, 3-trifluorovinyl-2-thienyl, 3-trichloromethylphen-1-yl (i.e., 3-CCl₃Ph-), 4-(3-bromoprop-1-yl)phen-1-yl (i.e., 4-BrCH₂CH₂CH₂Ph-), and the like. Further examples of aromatic radicals include 4-allyloxyphen-1-oxy, 4-aminophen-1-yl (i.e., 4-H₂NPh-), 3-aminocarbonylphen-1-yl (i.e., NH₂COPh-), 4-benzoylphen-1-yl, dicyanomethylidenebis(4-phen-1-yloxy) (i.e., —OPhC(CN)₂PhO—), 3-methylphen-1-yl, methylenebis(4-phen-1-yloxy) (i.e., —OPhCH₂PhO—), 2-ethylphen-1-yl, phenylethenyl, 3-formyl-2-thienyl, 2-hexyl-5-furanyl, hexamethylene-1,6-bis(4-phen-1-yloxy) (i.e., —OPh(CH₂)₆PhO—), 4-hydroxymethylphen-1-yl (i.e., 4-HOCH₂Ph-), 4-mercaptomethylphen-1-yl (i.e., 4-HSCH₂Ph-), 4-methylthiophen-1-yl (i.e., 4-CH₃SPh-), 3-methoxyphen-1-yl, 2-methoxycarbonylphen-1-yloxy (e.g. methyl salicyl), 2-nitromethylphen-1-yl (i.e., 2-NO₂CH₂Ph), 3-trimethylsilylphen-1-yl, 4-t-butyldimethylsilylphenl-1-yl, 4-vinylphen-1-yl, vinylidenebis(phenyl), and the like. The term “a C₃-C₁₀ aromatic radical” includes aromatic radicals containing at least three but no more than 10 carbon atoms. The aromatic radical 1-imidazolyl (C₃H₂N₂—) represents a C₃ aromatic radical. The benzyl radical (C₇H₇—) represents a C₇ aromatic radical.

The term “heteroaromatic ring” refers to a monovalent 5 or 6 membered monocyclic heteroaromatic ring containing carbon atoms and one or more heteroatoms selected from the group consisting of oxygen, nitrogen and sulfur. Representative examples of heteroaromatic groups are included thioazole, pyridinyl, furanyl, pyridyl, pyrrolyl, pyrazinyl, pyrimidinyl, pyridazinyl, thienyl (or thiophenyl), thiazolyl, imidazolyl, pyrazolyl, triazolyl, tetrazolyl, oxazolyl, isooxazolyl, oxadiazolyl, isothiazolyl, and thiadiazolyl.

As used herein the term “cycloaliphatic radical” refers to a radical having a valence of at least one, and comprising an array of atoms which is cyclic but which is not aromatic. As defined herein a “cycloaliphatic radical” does not contain an aromatic group. A “cycloaliphatic radical” may comprise one or more noncyclic components. For example, a cyclohexylmethyl group (C₆H₁₁CH₂—) is an cycloaliphatic radical which comprises a cyclohexyl ring (the array of atoms which is cyclic but which is not aromatic) and a methylene group (the noncyclic component). The cycloaliphatic radical may include heteroatoms such as nitrogen, sulfur, selenium, silicon and oxygen, or may be composed exclusively of carbon and hydrogen. For convenience, the term “cycloaliphatic radical” is defined herein to encompass a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylcyclopent-1-yl radical is a C₆ cycloaliphatic radical comprising a methyl group, the methyl group being a functional group which is an alkyl group. Similarly, the 2-nitrocyclobut-1-yl radical is a C₄ cycloaliphatic radical comprising a nitro group, the nitro group being a functional group. A cycloaliphatic radical may comprise one or more halogen atoms which may be the same or different. Halogen atoms include, for example; fluorine, chlorine, bromine, and iodine. Cycloaliphatic radicals comprising one or more halogen atoms include 2-trifluoromethylcyclohex-1-yl, 4-bromodifluoromethylcyclooct-1-yl, 2-chlorodifluoromethylcyclohex-1-yl, hexafluoroisopropylidene-2,2-bis (cyclohex-4-yl) (i.e., —C₆H₁₀C(CF₃)₂C₆H₁₀—), 2-chloromethylcyclohex-1-yl, 3-difluoromethylenecyclohex-1-yl, 4-trichloromethylcyclohex-1-yloxy, 4-bromodichloromethylcyclohex-1-ylthio, 2-bromoethylcyclopent-1-yl, 2-bromopropylcyclohex-1-yloxy (e.g. CH₃CHBrCH₂C₆H₁₀O—), and the like. Further examples of cycloaliphatic radicals include 4-allyloxycyclohex-1-yl, 4-aminocyclohex-1-yl (i.e., H₂NC₆H₁₀—), 4-aminocarbonylcyclopent-1-yl (i.e., NH₂COC₅H₈—), 4-acetyloxycyclohex-1-yl, 2,2-dicyanoisopropylidenebis(cyclohex-4-yloxy) (i.e., —OC₆H₁₀C(CN)₂C₆H₁₀—), 3-methylcyclohex-1-yl, methylenebis(cyclohex-4-yloxy) (i.e., —OC₆H₁₀CH₂C₆HoO—), 1-ethylcyclobut-1-yl, cyclopropylethenyl, 3-formyl-2-terahydrofuranyl, 2-hexyl-5-tetrahydrofuranyl, hexamethylene-1,6-bis(cyclohex-4-yloxy) (i.e., —O C₆H₁₀(CH₂)₆C₆H₁₀O—), 4-hydroxymethylcyclohex-1-yl (i.e., 4-HOCH₂C₆H₁₀—), 4-mercaptomethylcyclohex-1-yl (i.e., 4-HSCH₂C₆H₁₀—), 4-methylthiocyclohex-1-yl (i.e., 4-CH₃SC₆H₁₀), 4-methoxycyclohex-1-yl, 2-methoxycarbonylcyclohex-1-yloxy (2-CH₃OCOC₆H₁₀O—), 4-nitromethylcyclohex-1-yl (i.e., NO₂CH₂C₆H₁₀—), 3-trimethylsilylcyclohex-1-yl, 2-t-butyldimenthylcyclohex-1-yl, 4-trimethoxysilylethylcyclohex-1-yl (e.g. (CH₃O)₃SiCH₂CH₂C₆H₁₀—), 4-vinylcyclohexen-1-yl, vinylidenebis(cyclohexyl), and the like. The term “a C₃-C₁₀ cycloaliphatic radical” includes cycloaliphatic radicals containing at least three but no more than 10 carbon atoms. The cycloaliphatic radical 2-tetrahydrofuranyl (C₄H₇O—) represents a C₄ cycloaliphatic radical. The cyclohexylmethyl radical (C₆H₁₁CH₂—) represents a C₇ cycloaliphatic radical.

As used herein the term “aliphatic radical” refers to an organic radical having a valence of at least one consisting of a linear or branched array of atoms which is not cyclic. Aliphatic radicals are defined to comprise at least one carbon atom. The array of atoms comprising the aliphatic radical may include heteroatoms such as nitrogen, sulfur, silicon, selenium and oxygen or may be composed exclusively of carbon and hydrogen. For convenience, the term “aliphatic radical” is defined herein to encompass, as part of the “linear or branched array of atoms which is not cyclic” organic radicals substituted with a wide range of functional groups such as aLkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylpent-1-yl radical is a C₆ aliphatic radical comprising a methyl group, the methyl group being a functional group which is an alkyl group. Similarly, the 4-nitrobut-1-yl group is a C₄ aliphatic radical comprising a nitro group, the nitro group being a functional group. An aliphatic radical may be a haloalkyl group which comprises one or more halogen atoms which may be the same or different. Halogen atoms include, for example; fluorine, chlorine, bromine, and iodine. Aliphatic radicals comprising one or more halogen atoms include the alkyl halides trifluoromethyl, bromodifluoromethyl, chlorodifluoromethyl, hexafluoroisopropylidene, chloromethyl, difluorovinylidene, trichloromethyl, bromodichloromethyl, bromoethyl, 2-bromotrimethylene (e.g. —CH₂CHBrCH₂—), and the like. Further examples of aliphatic radicals include allyl, aminocarbonyl (i.e., —CONH₂), carbonyl, 2,2-dicyanoisopropylidene (i.e., —CH₂C(CN)₂CH₂—), methyl (i.e., —CH₃), methylene (i.e., —CH₂—), ethyl, ethylene, formyl (i.e. —CHO), hexyl, hexamethylene, hydroxymethyl (i.e. —CH₂OH), mercaptomethyl (i.e., —CH₂SH), methylthio (i.e., —SCH₃), methylthiomethyl (i.e., —CH₂SCH₃), methoxy, methoxycarbonyl (i.e., CH₃OCO—) , nitromethyl (i.e., —CH₂NO₂), thiocarbonyl, trimethylsilyl (i.e., (CH₃)₃Si—), t-butyldimethylsilyl, 3-trimethyoxysilypropyl (i.e., (CH₃O)₃SiCH₂CH₂CH₂—), vinyl, vinylidene, and the like. By way of further example, a C₁-C₁₀ aliphatic radical contains at least one but no more than 10 carbon atoms. A methyl group (i.e., CH₃—) is an example of a C₁ aliphatic radical. A decyl group (i.e., CH₃(CH₂)₉—) is an example of a C₁₀ aliphatic radical.

The term “heteroaryl” as used herein refers to aromatic or unsaturated rings in which one or more carbon atoms of the aromatic ring(s) are replaced by a heteroatom(s) such as nitrogen, oxygen, boron, selenium, phosphorus, silicon or sulfur. Heteroaryl refers to structures that may be a single aromatic ring, multiple aromatic ring(s), or one or more aromatic rings coupled to one or more non-aromatic ring(s). In structures having multiple rings, the rings can be fused together, linked covalently, or linked to a common group such as an ether, methylene or ethylene moiety. The common linking group may also be a carbonyl as in phenyl pyridyl ketone. As used herein, rings such as thiophene, pyridine, isoxazole, pyrazole, pyrrole, furan, etc. or benzo-fused analogues of these rings are defined by the term “heteroaryl.”

The term “aryl” is used herein to refer to an aromatic substituent which may be a single aromatic ring or multiple aromatic rings which are fused together, linked covalently, or linked to a common group such as an ether, methylene or ethylene moiety. The aromatic ring(s) may include phenyl, naphthyl, anthracenyl, and biphenyl, among others. In particular embodiments, aryls have between 1 and 200 carbon atoms, between 1 and 50 carbon atoms or between 1 and 20 carbon atoms.

The term “alkyl” is used herein to refer to a branched or unbranched, saturated or unsaturated acyclic hydrocarbon radical. Suitable alkyl radicals include, for example, methyl, ethyl, n-propyl, i-propyl, 2-propenyl (or allyl), vinyl, n-butyl, t-butyl, i-butyl (or 2-methylpropyl), etc. In particular embodiments, alkyls have between 1 and 200 carbon atoms, between 1 and 50 carbon atoms or between 1 and 20 carbon atoms.

The term “cycloalkyl” is used herein to refer to a saturated or unsaturated cyclic non-aromatic hydrocarbon radical having a single ring or multiple condensed rings. Suitable cycloalkyl radicals include, for example, cyclopentyl, cyclohexyl, cyclooctenyl, bicyclooctyl, etc. In particular embodiments, cycloalkyls have between 3 and 200 carbon atoms, between 3 and 50 carbon atoms or between 3 and 20 carbon atoms.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

### EXAMPLES

Examples 1-16 describe the syntheses of compounds of the invention and intermediates used in making them. All reagents were purchased from Aldrich Chemical Co., Milwaukee, Wis., USA unless other wise specified and were used without further purification. All compounds were characterized by ¹H-NMR and found to correspond to the structures shown.

### Example 1

### Synthesis of Compound 1

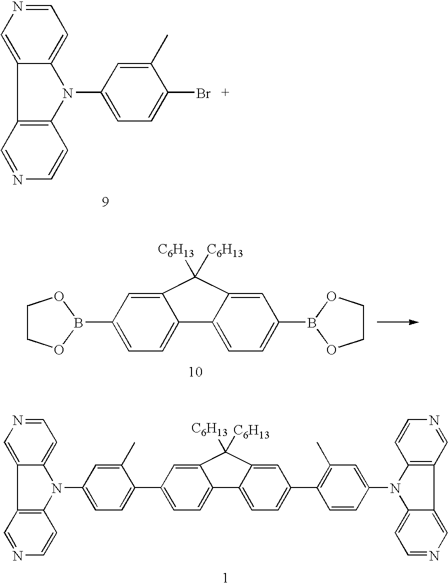

4-bromo-3-methyl phenyl diaza-carbazole(compound 9, 0.338 g, 0.1 mmol) and 0.25 g (0.05 mol) of 9,9-dihexylfluorene-2,7-diboronic acid bis(ethylglycol) ester (compound 10) were placed into a 50 mL of three neck flask. 10 mL of dioxane and 10 mL of K₂CO₃ (2N aqueous solution) were added to the flask. The mixture was evacuated and filled with argon three times and then a very small pinch of Pd(PPh₃)₄ was added. The mixture was stirred and evacuated and filled with argon three times. The flask was immersed in an oil bath and refluxed overnight. After the solution was cooled to room temperature, the solvent was removed by roto-evaporation to afford crude product. The crude product was re-dissolved into methylene choloride and water mixture (1:1, 50 mL). The organic and aqueous layer was separated. The organic layer was washed with water (10 mL×2) and brine (10 ml×1). The mixture was concentrated and loaded onto an AlO₂ basic sample cartridge. The sample was eluted out using THF with 2% methanol using combi-flash system. The fractions was concentrated and chilled in a refrigerator overnight, this collected 0.1136 g of the product. ¹H NMR (CDCl₃, δ) 9.46 (s, 4H), 8.62 (d, 4H), 7.90 (d, 2H), 7.64 (d, 2H), 7.54 (b, 2H), 7.49 (d, 4H), 7. 45 (dd, 6H), 2.48 (s, 6H), 2.10 (m, 4H), 1.15 (m, 12H), 0.81 (tm, 10H). Maldi (M+=849.12)

### Example 2

### Synthesis of Compound 10

A solution of 9,9-dihexylfluorene-2,7-bis-boronic acid (2.9 g, 6.9 mmol) and ethylene glycol (1.7 g, 27.5 mmol) in chloroform (20 ml) was heated at reflux for 1 hour. The cooled mixture was diluted with saturated sodium chloride and the organic layer was passed through a cone of anhydrous CaSO₄ (Drierite). Evaporation of solvent afforded 3 g of a solid mass that was recrystallized from acetonitrile to afford 2.34 g (72%) of the product as a white solid. ¹H NMR (CD₂Cl₂) δ 7.8 (m, 6, ArH), 4.48 (s, 8, OCH₂), 2.05 (m,4,Ar₂CCH₂C₅H₁₁), 1.1 (m,12,aliphatic CH₂) 0.75 (t,6,CH₂CH₃) and 0.60 ppm (m,4,penultimate CH₂)

### Example 3

### Synthesis of Compound 2

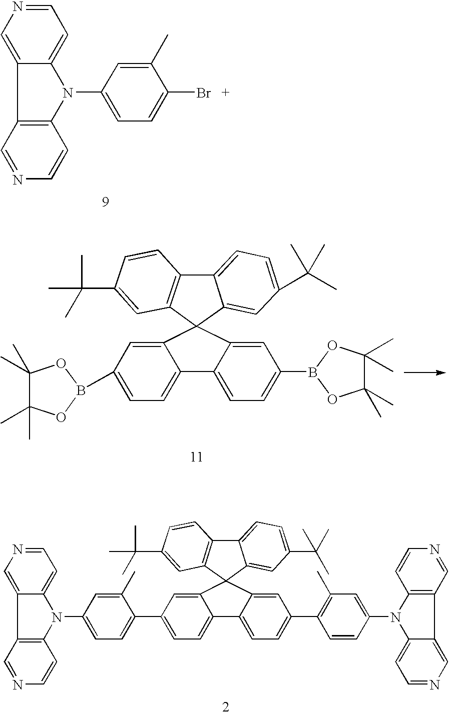

4-Bromo-3-methyl phenyl diaza-carbazole (compound 9, 0.3486 g, 1.031 mmol) and 0.3422 g (0.5028 mmol) of 2′,7′-di-t-butyl spirofluorene-2,7-diboronic acid bis(neopentyl diol) ester (compound 11) were placed into a 50 mL of three neck flask. 10 mL of dioxane and 10 mL of K₂CO₃ (2N aqueous solution) were added to the flask. The mixture was evacuated and filled with argon three times and then a very small pinch of Pd(PPh₃)₄ was added. The mixture was stirred and evacuated and filled with argon three times. The flask was immersed in an oil bath and refluxed overnight. After the solution was cooled to room temperature, the solvent was removed by roto-evaporation to afford crude product. The crude product was re-dissolved into methylene choloride and water mixture (1:1, 50 mL). The organic and aqueous layer was separated. The organic layer was washed with water (10 mL×2) and brine (10 mL×1). The mixture was concentrated and loaded onto an AlO₂ basic sample cartridge. The sample was eluted out using THF with 2% methanol using combi-flash system. The fractions was concentrated and chilled in a refrigerator overnight, this collected 0.10 g of product.

### Example 4

### Synthesis of Compound 11

A solution of 2,7-dibromo-2′,7′-di-tert-butylspirofluorene (5.86 g, 10 mmol) in anhydrous diethylether (100 ml) was cooled to −78° C. and treated dropwise with a solution of n-butyllithium (1.6 M in hexane, 20.8 mmol). The stirred mixture was allowed to warm to ambient temperature and stirring was continued for an additional hour. The resulting slurry was re-cooled to −78 ° C and treated with isopropoxypinacol borate (4.65 g, 25 mmol). The mixture was again allowed to warm to ambient temperature and was stirred an additional 3 hours. The mixture was quenched by addition of water (25 ml) and toluene (50 ml) was added. Combined organic washings were dried (CaSO₄) and solvent was removed under vacuum to afford a white solid that was recrystallized from toluene to afford 3.7 g (55%) of the bis borate. ¹H NMR (CD₂Cl₂) δ 7.9-6.6 (m,12,ArH), 1.30 (s,24,borate CH3's) and 1.20 ppm (s,18,tBu).

### Example 5

### Synthesis of Compound 3

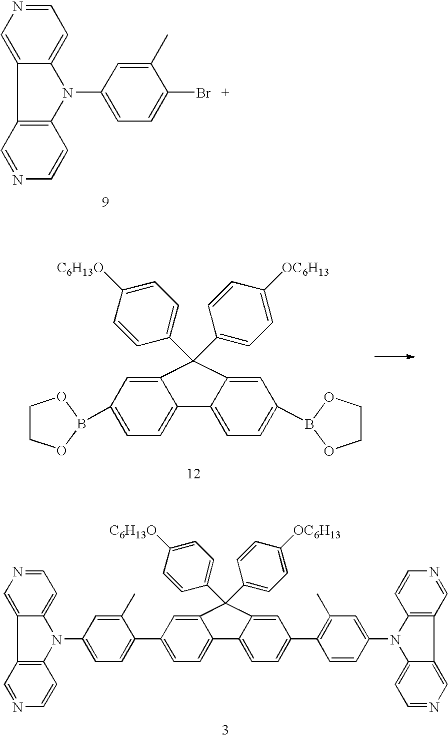

N-(3-methyl-4-bromo phenyl) diaza carbazole (compound 9, 1.314 g, 3.88 mmol) and [9,9-bis[4-(hexyloxy)phenyl]-fluorene-2,7-diyl]bis-1,3,2-dioxaborolane (compound 12, 1.2793 g, 1.94 mmol) were weighed out into a 100 mL of three neck round bottom flask. To this flask, 40 mL of dioxane and 40 mL of K₂CO₃ (2N) were added. The mixture was degassed using argon for 30 minutes. Then a pinch of Pd(PPh₃)₄ was added. The reaction was heated under reflux for overnight. The entire mixture was evaporated to dryness using roto-evaporation. The residue was re-dissolved in a 200 mL of CH₂Cl₂ and 100 mL of water mixture. The organic layer and aqueous layer was separated. The organic layer was washed with water (100 mL×2) and brine (100 ml×1). After dried over Na₂SO₄, the solvent was removed and the crude product was separated using alumina basic column with THF/hexanes as the eluting solvent. 0.96 g of product was obtained. ¹H NMR (CDCl₃, δ) 9.46 (s, 4H), 8.62 (d, 4H), 7.94 (d, 2H), 7.55 (d, 2H), 7.49 (s, 2H), 7. 47 (dd, 2H), 7.45 (s, 2H), 7.43(s+d, 4H), 7.39 (d, 2H), 7.24 9d, 2H), 3.91 (t, 4H), 2.40 (s, 6H), 1.76 (m, 4H), 1.44 (b, 4H), 1.33 (m, 8H), 0.90 (s, 6H). Maldi (M+ 1033.6000)

### Example 6

### Synthesis of Compound 4

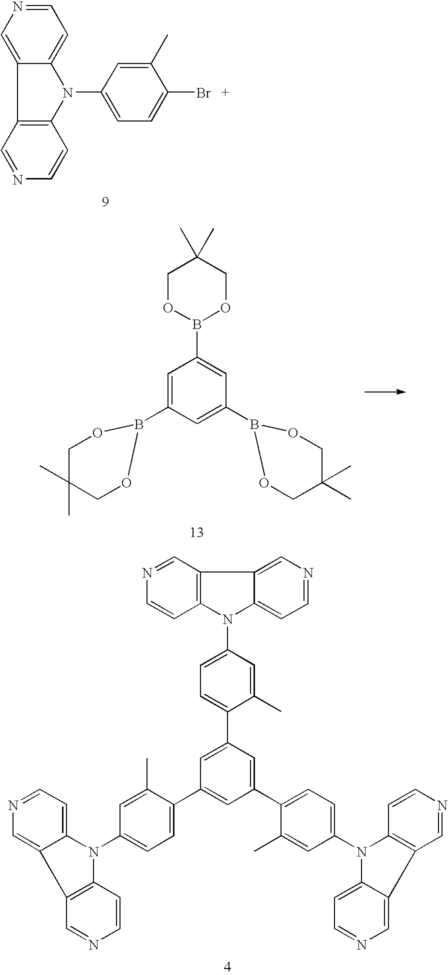

4-bromo-3-methyl phenyl diaza-carbazole (compound 9, 0.6216 g, 1.83 mmol) and 0.2377 g (0.5743 mmol) of 1,3,5-benzene triboronic acid bis(neopentyl diol) ester (compound 13) were placed into a 50 mL of three neck flask. 25 mL of dioxane and 10 mL of K₂CO₃ (2N aqueous solution) were added to the flask. The mixture was evacuated and filled with argon three times and then a very small pinch of Pd(PPh₃)₄ was added. The mixture was stirred and evacuated and filled with argon three times. The flask was immersed in an oil bath and refluxed overnight. After the solution was cooled to room temperature, the product was precipitated out. The solid product was collected by centrifuge and washed with water acetone couple of times. Maldi (M+850.6234), ¹H NMR (CDCl₃, δ) 9.49 (s, 6H), 8.65 (d, 6H), 7.70 (d, 3H), 7.59 (s, 3H), 7.53 (m, 3H), 7.49 (dd, 3H), 7.44 (d, 6H), 2.27 (s, 9H).

### Example 7

### Synthesis of Compound 13

Step 1. A flame dried 100 ml flask equipped with an overhead stirrer was charged with small Mg turnings (3.65 g, 150 g-atom), chlorotrimethylsilane (16 g, 140 mmol), 1,2-dibromoethane (100 mg) and anhydrous THF (40 ml). To the stirred mixture was added over 1 hour 1,3,5-tribromobenzene (12.5 g, 39.7 mmol). The reaction mixture began to reflux almost immediately on addition of the aryl bromide. When the exothermic reaction subsided the mixture was heated at reflux for 16 hours. On cooling, the mixture solidified to a hard cake. The liquid phase was decanted from the solid MgBr₂ and the solids were washed repeatedly with ether. Combined ether extracts were washed with water and brine then stripped to afford 11.4 g of a dark oil. The oil was chromatographed on ˜100 g of silica gel eluted with hexane.

Step 2. A flame-dried 10 ml flask was charged with purified tris-trimethylsilylbenzene prepared as described in step 1, (395 mg, 1.34 mmol). To this was added BBr₃ (700 μl, 1.85 g, 7.4 mmol). The resulting mixture was heated at reflux (˜90° C.) for 35 hour. The mixture was cooled to room temperature and excess BBr₃ was removed by vacuum distillation. The residue was diluted with hexane and allowed to stand at room temperature overnight. Water was added cautiously followed by a mixture of methanol and neopentyl glycol. The mixture was stripped to near dryness and the residue was stirred with methanol to dissolve excess neopentyl glycol and deposit the product as a white solid (84%). ¹H NMR (CH₂Cl₂) δ 8.35 (s,3,ArH), 3.80 (s,12,borate OCH₂) and 1.0 ppm (s,18,borate CH₃'s)

### Example 8

### Synthesis of Compound 5

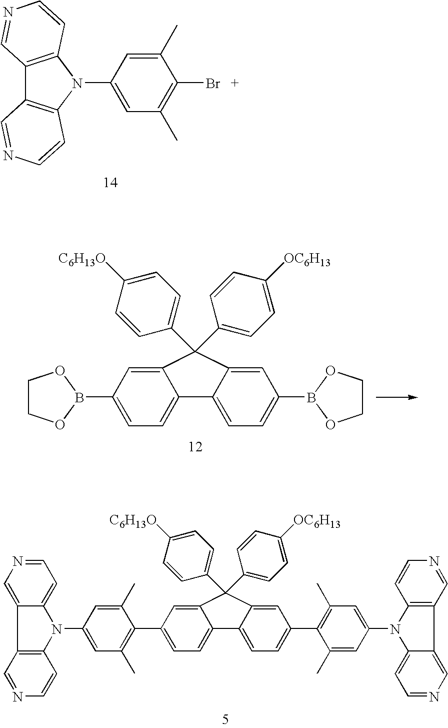

N-(3,5-dimethyl-4-bromo phenyl) diaza carbazole (compound 14, 1.21 g, 3.4 mmol) and [9,9-bis[4-(hexyloxy)phenyl]-fluorene-2,7-diyl]bis-1,3,2-dioxaborolane (compound 12, 0.7539 g, 1.14 mmol) were weighed out into a 500 mL of three neck round bottom flask. To this flask, 34 mg of Pd(OAc)₂ and 220 mg of 2-dicyclohexylphosphino-2′,6′-dimethoxybiphenyl were added, followed by 200 mL of toluene. The solution was degassed using a stream of argon. In a separate flask, 3.7 g of water and 3.7 g of tetraethylamonium hydroxide were combined and degassed using a stream of argon for 15 minuntes. The base solution was added to the toluene solution in a drop wise fashion. The toluene solution was brought to 80° C. under argon and stirred overnight. The second day, upon cooling, the solution was filtered and evaporated to dryness. The residue was separated using alumina basic column with THF as the eluting solvent. 0.6 g of product was obtained. ¹H NMR (CDCl₃, δ) 9.49 (s, 4H), 8.65 (d, 4H), 7.98 (d, 2H), 7.45(d, 4H), 7.33 (s, 2H), 7.31(dd, 2H), 7.27(s, 4H), 7.22(d, 4H), 3.92 (t, 4H), 2.16 (s, 12H), 1.76 (m, 4H), 1.44 (b, 4H), 1.33 (m, 8H), 0.90 (s, 6H). Maldi (M+=1061.7932)

### Example 9

### Synthesis of Compound 14

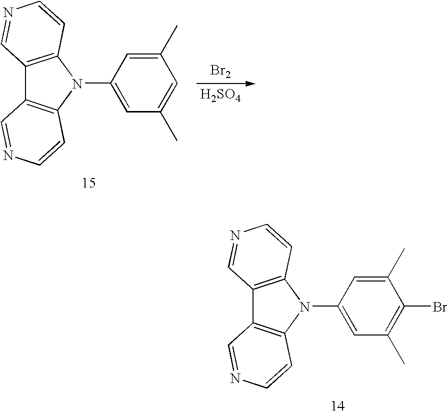

N-(3,5-dimethyl phenyl) diaza carbazole (compound 15, 1.3 g, 4.7 mmol) was gradually added to 5 mL of concentrated H₂SO₄, which was chilled in ice water, 0.485 mL of bromine was added over 10 minutes. The mixture was gradually warmed up to room temperature and refluxed for 24 hours. The reaction mixture was poured into 40 mL of ice water and diluted by THF (20 mL). After the solution was neutralized by Na₂CO₃ (8.8 g), the organic layer was separated and removed. The residue was extracted with 30 mL of THF and 2 mL of methanol mixture. The insoluble material was discarded. THF and methanol mixture was concentrated to afford crude product as a mixture of two isomers. After crystallization from acetonitrile, only desired symmetrical mixture was collected. ¹H NMR (CDCl₃, δ) 9.47 (s, 2H), 8.62 (d, 2H), 7.34 (dd, 2H), 7.25 (s, 2H), 2.56(s. 6H).

### Example 10

### Synthesis of Compound 15

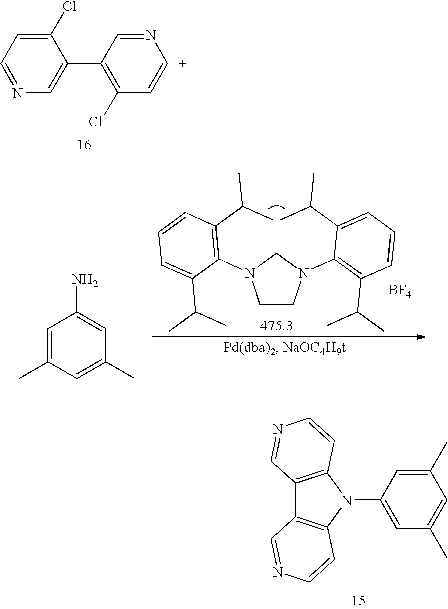

To 9.38 mL of 1,2-dimethoxyethane (DME), Pd(dba)₂ (0.495 g, 0.08735 mmol) and ITF (0.411 g, 0.08735 mmol) were added and the mixture was stirred at 45° C. for 20 minutes. To this mixture, dimethyl aniline (3.53 g, 0.029 mol), compound 16 (6.47 g, 0.03 mol), tBuONa (7.18 g, 0.0745 mol), and DME (84 mL) were added. This mixture was refluxed overnight. The next day, the solution was cooled at 50° C. Saturated NaCl aqueous solution (36 mL) was added, the insoluble solid was separated by filtration, then the residue was washed with THF (50 mL). The organic layer of filtrate was separated; the solvent was removed under reduced pressure. The first crop of product was obtained by extracting the solid using ethylacetate. A second crop of product was collected by adding hexane into ethyl acetate solution. Combined to give 85% of yield.

### Example 11

### Synthesis of Compound 16

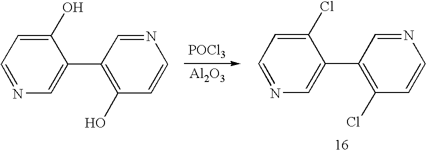

2,2′-Dihydroxy-5,5′-bipyridine (92.67 g, 0.14 mmol) was added to 8.5 mL of phosphorus oxy-chloride and was refluxed for an hour. Then excess amount of phosphorus oxy-chloride was removed under reduced pressure. The residue was diluted by toluene (7.7 mL), and was slowly added ice water within 60° C. for an hour. To this mixture, 42 mL of ethyl acetate was added. The mixture was neutralized using K₂CO₃ until the PH was basic. The precipitate was removed by filtration and the filtrate was separated. Organic layer was combined and washed with equal amount of water and saturated brine solution. The solvent was removed and the solids were redissolved into ethyl acetate/toluene (1/4) mixture and 18 g of Al₂O₃ was added. The mixture was stirred for 40 minutes, then Al₂O₃ was filtered and solvent was removed. The crude product was re-crystallized from hexanes to obtain 1.7 g of desired product.

### Example 12

### Preparation of Compound 6

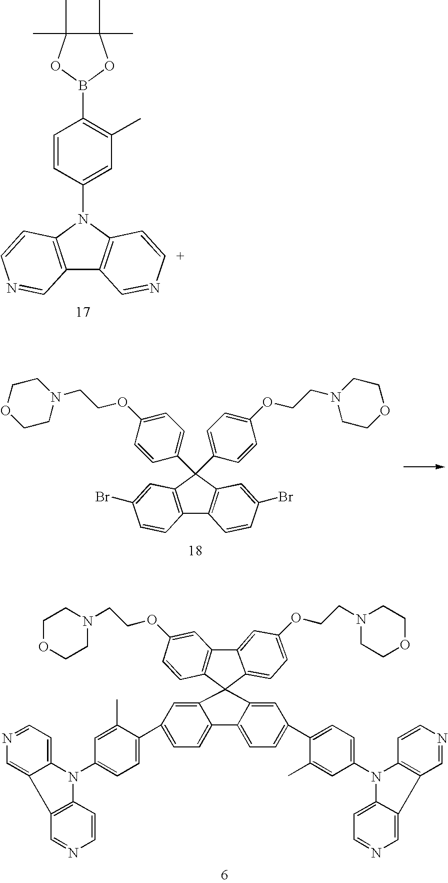

A degassed mixture of compound 17 (385 mg, 1.0 mmol), compound 18 (367 mg, 0.5 mmol), Pd(OAc)₂ (4.5 mg, 0.02 mmol), tri-o-tolylphosphine (21.4 mg, 0.07 mmol), tetraethylammonium hydroxide (7.4 g of a 10% aqueous solution) and toluene (20 ml) was heated under nitrogen for 18 hours at 70° C. The cooled mixture was diluted with toluene, filtered through Celite and transferred to a separatory funnel. The organic phase was washed with water (2×50 ml) and saturated NaCl (1×50 ml). Removal of solvent afforded a residue that was chromatographed on silica gel eluted with a methanol/methylene chloride gradient. The product, compound 6, was obtained as a colorless foam.

### Example 13

### Preparation of Compound 7

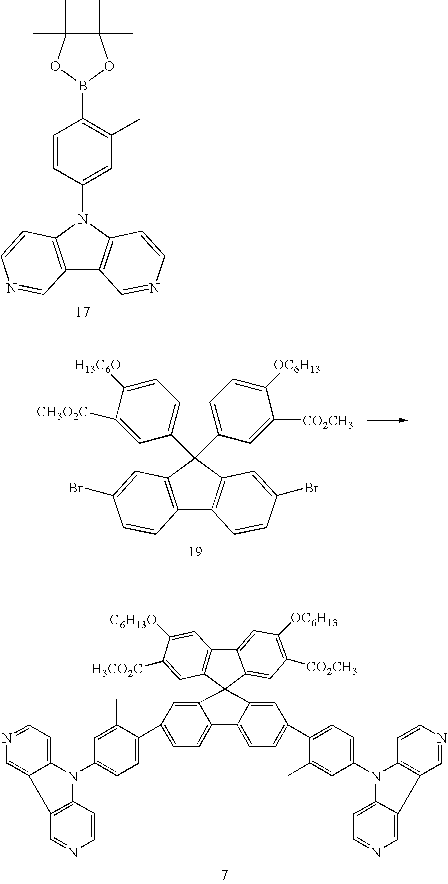

A degassed mixture of compound 17 (385 mg, 1.0 mmol), compound 19 (367 mg, 0.5 mmol), Pd(OAc)₂ (4.5 mg, 0.02 mmol), tri-o-tolylphosphine (21.4 mg, 0.07 mmol), tetraethylammonium hydroxide (7.4 g of a 10% aqueous solution) and toluene (20 ml) was heated under nitrogen for 18 hours at 70° C. The cooled mixture was diluted with toluene, filtered through Celite and transferred to a separatory funnel. The organic phase was washed with water (2×50 ml) and saturated NaCl (1×50 ml). Removal of solvent afforded a residue that was chromatographed on silica gel eluted with a methanol/methylene chloride gradient. The product, compound 7, was obtained as a colorless foam.

### Example 14

### Preparation of Compound 17

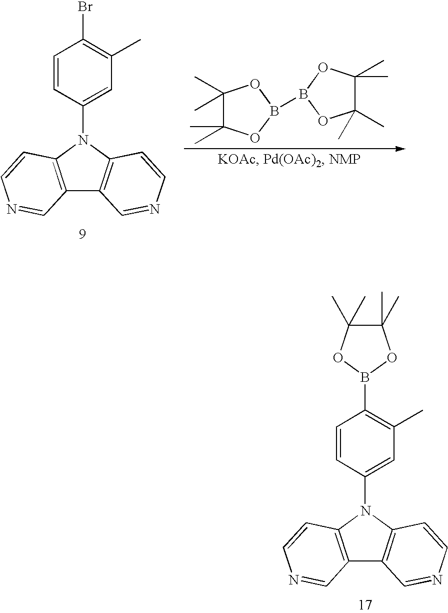

A degassed solution of compound 9 (3.98 g, 11.76 mmol), bis-pinacolatodiborane (4.48 g, 17.6 mmol), potassium acetate (5.19 g, 52.9 mmol) and Pd(OAc)₂ (0.26 g, 1.176 mmol) in N-methylpyrrolidinone (100 ml) was heated under nitrogen at 128° C. for 16 hours. The cooled mixture was filtered through silica gel. The filtrate was diluted with ethyl acetate (150 ml) and transferred to a separatory funnel. The solution was washed with cold 1N HCl (3×150 ml), water (1×150 ml) and saturated NaCl (1×100 ml). Removal of solvent afforded a residue that was chromatographed on silica gel eluted with 50-100% ethyl acetate-hexane to afford 1.4 g (30%) of the desired product.

### Example 15

### Synthesis of Compound 18

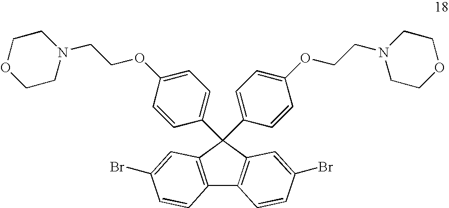

A 250 ml flask fitted with a magnetic stirrer and a distillation head was charged with 2,7-dibromo-9,9-bis-4-hydroxyphenylfluorene (5.08 g, 10 mmol) and toluene (130 ml). The flask and solids were azeotropically dried by distillative removal of approximately 100 ml of toluene. The flask was then charged with DMF (150 ml), K₂CO₃ (5.80 g, 42 mmol) and N-2-chloroethylmorpholine (3.91 g, 21 mmol). This mixture was heated at 90° C. for 4 days. The cooled mixture was poured into 400 g of ice water and the residue was extracted with 3×200 ml of ethyl ether. The combined ether washes were washed with water (3×150 ml) and brine (1×100 ml), then stripped to afford an oil that was triturated with cold ether to afford 8 g of a fluffy white powder that was chromatographed on 80 g of neutral alumina to afford the product as a white solid. ¹H NMR (CD₂Cl₂) δ 7.7-7.4 (m,6,fluorene-H's), 4.08 (t,4,ArOCH₂), 3.68 (t,8,morpholine-OCH₂), 2.78 (t,4,N—CH₂CH₂Oar) and 2.58 ppm (t,8, morpholine-N—CH₂'s)

### Example 16

### Synthesis of Compound 19

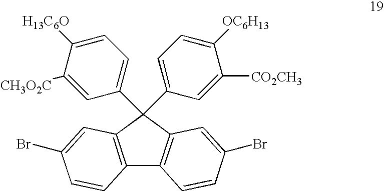

Step 1. Preparation of 2,7-dibromo-9,9-bis-(4-hydroxy-3-carbomethoxy)phenylfluorene. A mixture of 2,7-dibromofluorenone (16 g, 4.76 mmol), methyl salicylate (76 g, 500 mmol), methanesulfonic acid (100 ml) and an ion exchange resin having bound mercapto groups (6.0 g) was stirred mechanically at 70° C. for 16 hours. The cooled mixture was poured into 1 liter of ice water, diluted with ˜650 ml of ethyl acetate, then filtered to remove the ion exchange resin. The aqueous phase was discarded and the organic phase was washed with water (2×300 ml) and brine (1×300 ml), then passed through a cone of anhydrous CaSO₄. Removal of solvent under vacuum afforded a residue that was diluted with hexane and chilled to deposit a white solid that was recrystallized from toluene to afford 17.4 g (60%) of the product as a white solid. ¹H NMR (CH₂Cl₂) δ 10.8 (s,2,ArOH), 8.0-6.9 (m,12,ArH), and 3.90 (s,6,ArCO₂CH₃).

Step 2. A solution of 2,7-dibromo-9,9-bis-(4-hydroxy-3-carbomethoxy)phenylfluorene (4.68 g, 7.5 mmol), n-hexylbromide (3.2 g, 19 mmol), K₂CO₃ (4.0 g, 28.9 mmol) and tetrabutylammonium bromide (50 mg) in N,N-dimethylformamide (125 ml) was heated and stirred at 100° C. After 21 hours an additional 0.5 g of n-hexylbromide and 0.5 g of K₂CO₃ was added and heating was continued for an additional 5 hours. The cooled mixture was poured into 400 ml of cold HCl (0.5 N). The acidified mixture was extracted with 2×200 ml of ethyl acetate and the combined organic extracts were washed with water (3×300 ml) and brine (1×200 ml). The dried extracts (using CaSO₄) were stripped to dryness under vacuum and the residue, a light amber oil, was crystallized from methanol to afford 1.6 g (27%) of the product as a white solid. Additional product was obtained from the mother liquors. ¹H NMR (CH₂Cl₂) δ 7.75-6.80 (m,12,ArH), 4.05 (t,4,ArOCH₂), 3.80 (s,6,ArCO₂CH₃), 1.80 (m,4,ArOCH₂CH₂), 1.60-1.35 (m,12, ArOCH₂CH₂CH₂CH₂CH₂CH₃) and 0.98 ppm (t,6, ArOCH₂CH₂CH₂CH₂CH₂CH₃).

While only certain features of the invention have been illustrated and described herein, many modifications and changes will occur to those skilled in the art. It is, therefore, to be understood that the appended claims are intended to cover all such modifications and changes as fall within the true spirit of the invention.

1. An optoelectronic device comprising:
a cathode;
an electron-transporting layer comprising a compound of formula I; 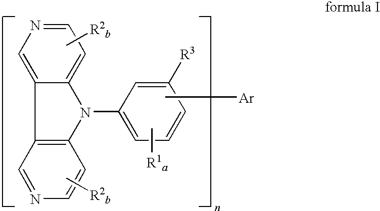
a light emitting-layer; and
an anode;
wherein
R¹ and R² are independently at each occurrence, hydrogen, a C₁-C₂₀ aliphatic radical, a C₃-C₂₀ aromatic radical, or a C₃-C₂₀ cycloaliphatic radical;
R³ is H or alkyl;
a and b are, independently at each occurrence 0, or an integer ranging from 1 to 3;
n is 2 or 3; and
Ar is an aryl.

2. The optoelectronic device of claim 1, wherein the compound of formula I is

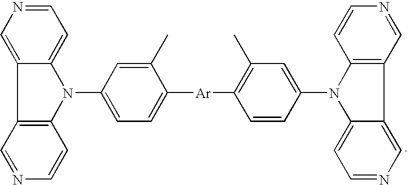

3. The optoelectronic device of claim 1, wherein Ar is 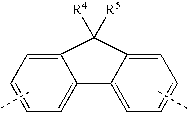
wherein R⁴ and R⁵ are independently a C₁-C₂₀ aliphatic radical, a C₃-C₂₀ aromatic radical, a C₃-C₂₀ cycloaliphatic radical, or R⁴ and R⁵, taken together, form a 10- to 13-membered bicyclic or tricyclic aromatic or heteroaromatic ring system.

4. The optoelectronic device of claim 1, wherein Ar is 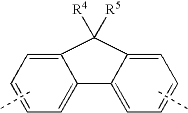
wherein R⁴ and R⁵ are independently a C₆-C₂₀ aliphatic radical, a C₆-C₂₀ aromatic radical, a C₆-C₂₀ cycloaliphatic radical, or R⁴ and R⁵, taken together, form a 10- to 13-membered bicyclic or tricyclic aromatic or heteroaromatic ring system.

5. The optoelectronic device of claim 1, wherein the compound of formula I is

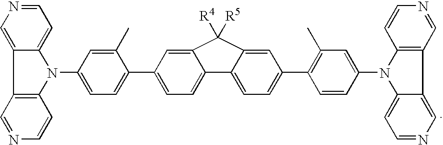

6. The optoelectronic device of claim 1, wherein Ar is 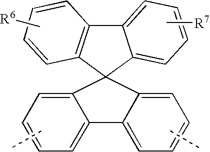
wherein R⁶ and R⁷ are independently a C₁-C₂₀ aliphatic radical, a C₃-C₂₀ aromatic radical, or a C₃-C₂₀ cycloaliphatic radical.

7. The optoelectronic device of claim 1, wherein Ar is 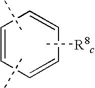
and R⁸ is independently at each occurrence a direct bond or an alkyl and c is an integer ranging from 2 to 6.

8. The optoelectronic device of claim 1, wherein Ar is selected from

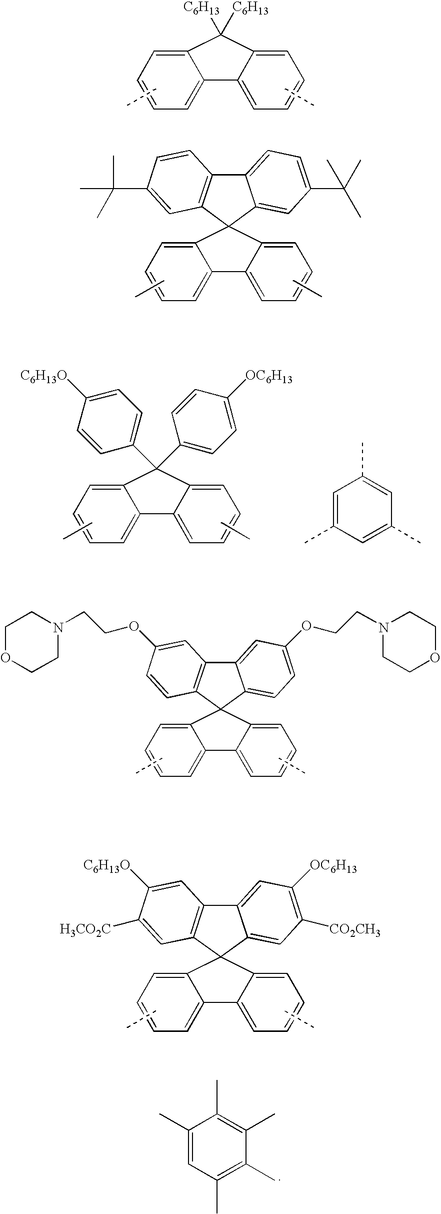

9. The optoelectronic device of claim 1, wherein Ar is selected from

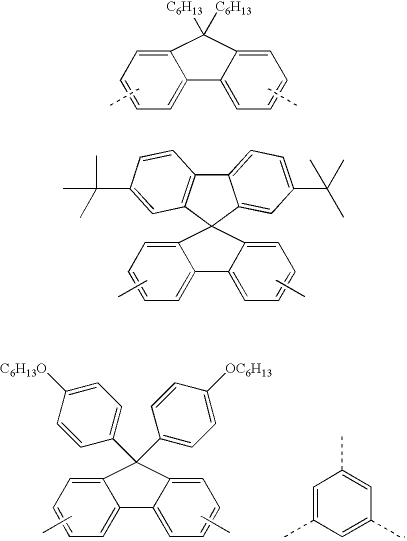

10. The optoelectronic device of claim 1, wherein R³ is methyl.

11. The optoelectronic device of claim 1, wherein the compound is of formula

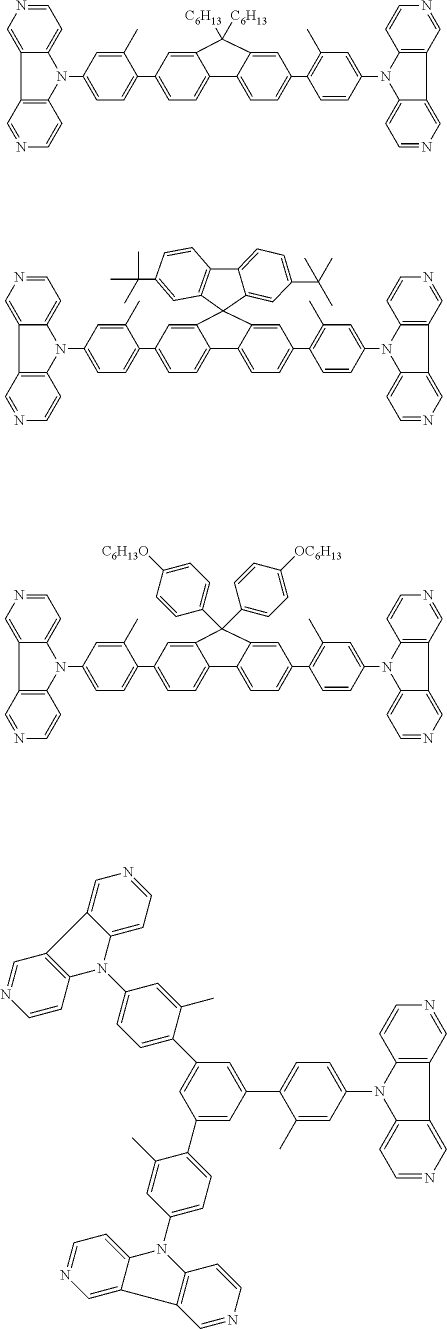

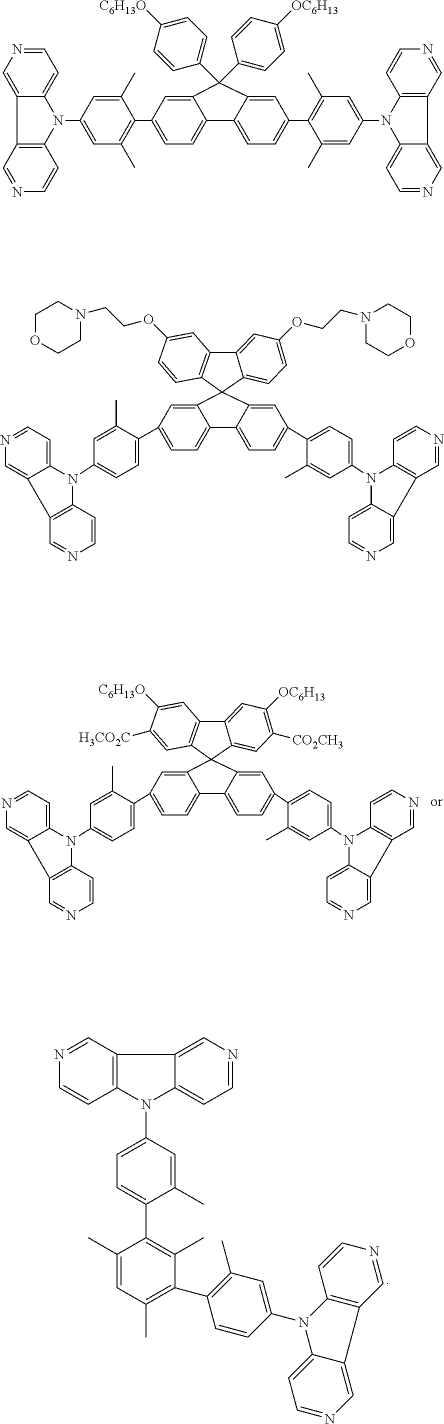

12. The optoelectronic device of claim 1, wherein the compound is of formula

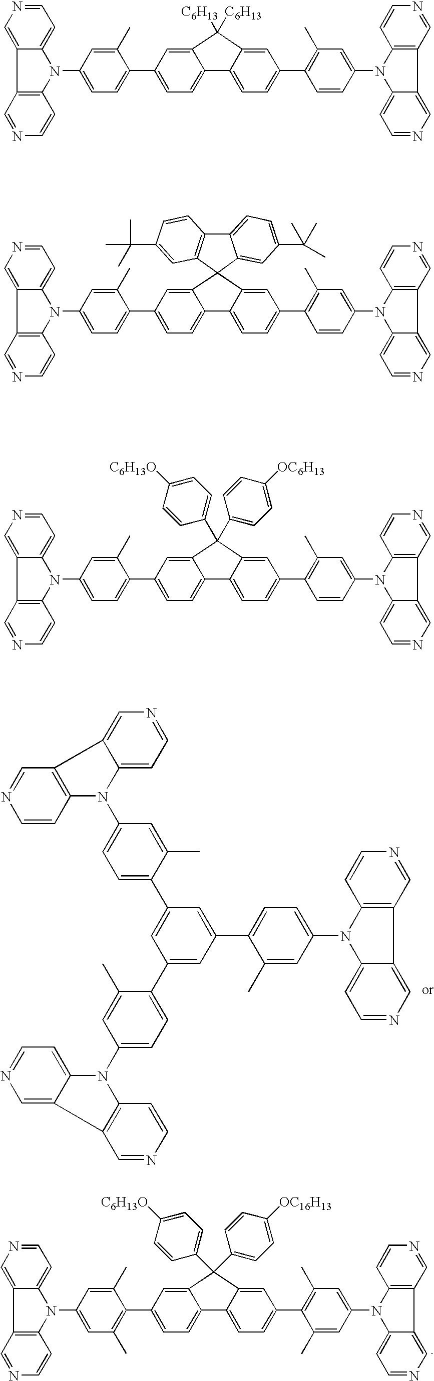

13. A compound of formula I: 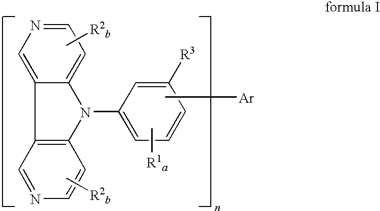
wherein
R¹ and R² are independently at each occurrence, hydrogen, a C₁-C₂₀ aliphatic radical, a C₃-C₂₀ aromatic radical, or a C₃-C₂₀ cycloaliphatic radical;
R³ is H or alkyl;
a and b are, independently at each occurrence 0, or an integer ranging from 1 to 3;
n is 2 or 3; and
Ar is an aryl.

14. The compound of claim 13, wherein Ar is 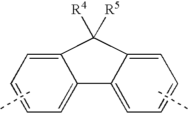
wherein R⁴ and R⁵ are independently a C₆-C₂₀ aliphatic radical, a C₆-C₂₀ aromatic radical, a C₆-C₂₀ cycloaliphatic radical, or R⁴ and R⁵, taken together, form a 10- to 13-membered bicyclic or tricyclic aromatic or heteroaromatic ring system.

15. The compound of claim 13, being of formula

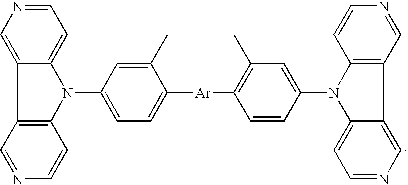

16. The compound of claim 13, wherein Ar is 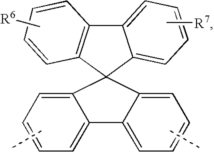
wherein R⁶ and R⁷ are independently a C₁-C₂₀ aliphatic radical, a C₃-C₂₀ aromatic radical, or a C₃-C₂₀ cycloaliphatic radical.

17. The compound of claim 1, wherein Ar is 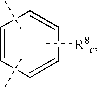
wherein R⁸ is independently at each occurrence a direct bond or an alkyl and c is an integer ranging from 2 to 6.

18. The compound of claim 13, wherein Ar is selected from

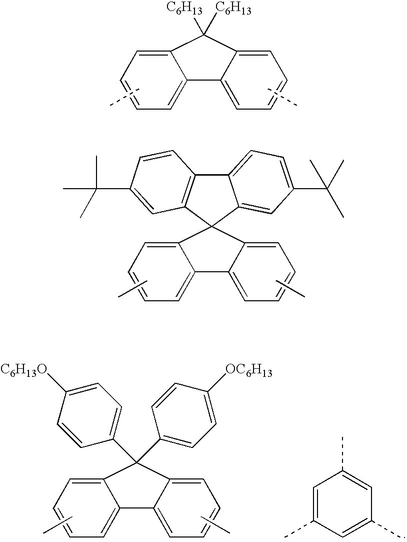

19. The compound of claim 13, wherein R³ is methyl.

20. The compound of claim 13, being of formula 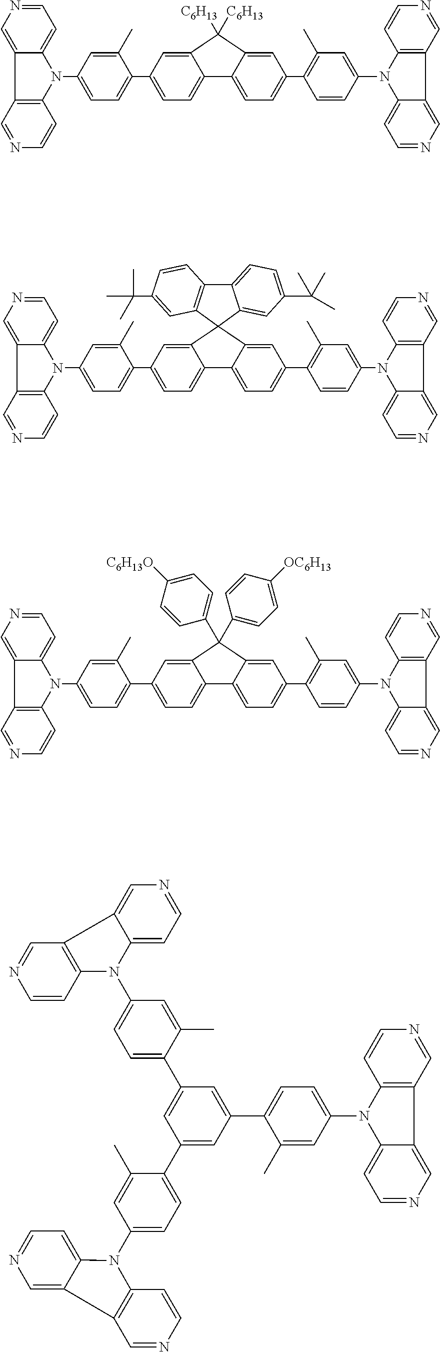 [chemical structure]
